(19) United States
(12) Patent Application Publication
(10) Pub. No.: US 2024/0336580 A1
(43) Pub. Date: Oct. 10, 2024

(54) IONIC SCINTILLATORS

(71) Applicants: CENTRE NATIONAL DE LA RECHERCHE SCIENTIFIQUE, PARIS (FR); UNIVERSITÉ DE STRASBOURG, STRASBOURG (FR)

(72) Inventors: Laurent DOUCE, STRASBOURG (FR); Nicolas DEL GIUDICE, ACHENHEIM (FR); Alexander KANE, SARASOTA, FL (US); Louise STUTTGE, STRASBOURG (FR)

(21) Appl. No.: 18/580,545

(22) PCT Filed: Jul. 19, 2022
(86) PCT No.: PCT/EP2022/070188

§ 371 Date: Jan. 18, 2024

(30) Foreign Application Priority Data
Jul. 20, 2021 (FR) FR2107798

(51) Int. Cl. C07D 263/32; G01T 1/203; G01T 1/204; G01T 3/06

(52) U.S. Cl. CPC C07D 263/32; G01T 1/2033; G01T 1/2042; G01T 3/06

(57) ABSTRACT

The invention relates to a compound having one of the following ionic chemical structures (I), “R” represents an “alkyl” or “O-alkyl” group optionally comprising one or more unsaturations, as a linear or branched chain, of 1 to 30 carbon atoms, optionally substituted; R1, R2, R3, R4, R5, R6, R7, R8, R9, R10, R11 and R12 represent, independently of one another, an atom or a group of atoms; “(Het)aryl” independently represents an aryl or heteroaryl group; and “A” represents an anion.

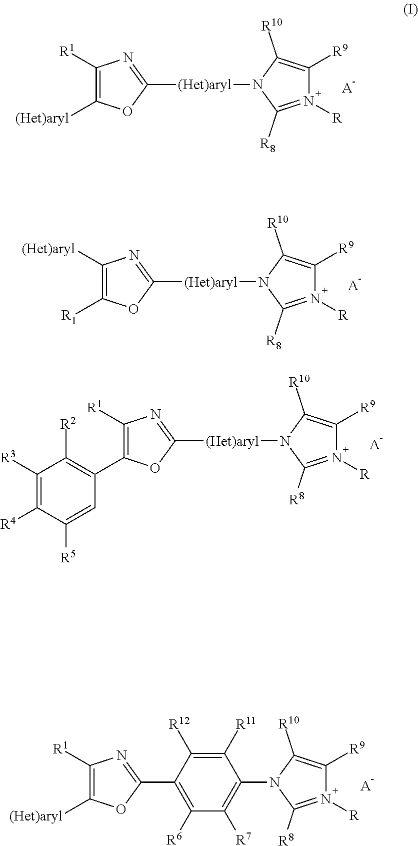

The present invention relates to a new family of discriminating (saline) ionic molecules for nuclear, neutron and gamma radiations in particular and a preparation method thereof. In particular, these molecules can be used for the detection of neutrons at all energies ranging from thermal energies to energies in the range of GeV. These materials can be used in all applications requiring the detection of neutrons both in fundamental research and in applied fields such as radioprotection, dosimetry, fissile material monitoring in nuclear wastes, diagraphy, medical imaging, etc.

### PRIOR ART

Scintillators are based on the fact that, upon passage of an ionising radiation, some materials emit light.

There are three types of scintillators classified according to the scintillation mechanisms involved:
organic: fluorescence process based on the structure of the molecule
inorganic: scintillation mechanism based on the crystalline structure
gas: energy of the incident particle transmitted to the atoms of the gas.

The quality of a scintillator is characterised by some properties:
high efficiency in the conversion of the kinetic energy of the incident particle into emitted light
linear conversion
transparency of the scintillator to its own light emission in order to avoid re-absorption of the signal
short response times in order to enable high count rates
good optical qualities in order to consider designing large-sized scintillators
a refractive index close to that of glass in order to couple the scintillator to a photodetector converting the light into an electrical signal
emission wavelength above 400 nm, a region of optimal quantum yield of photocathodes.

Organic and inorganic scintillators are most commonly used. While inorganic scintillators have a better light response and good linearity, their response time is much longer than that of organic scintillators, less light but very fast and less expensive.

The new material disclosed in this invention, as well as those of the patent applications or patents WO 2010/004228 (US 2011-0303850, EP 09784508) and WO 2014/147078 (U.S. Pat. No. 10,266,498, EP 2976330) is an ionic organic compound.

In the organic scintillators, the absorption of energy provided by the ionising particle causes the excitation of the p delocalised electrons of the molecules of the medium, covering a series of singlet electron states of the spin S=0 (S₀, S₁, S₂, . . . ), as well as the vibrational states (S₀₀, S₀₁, . . . , S₁₀, S₁₁, . . . , S₂₀, S₂₁, . . . ) of these electronic states. The upper singlet states will be de-excited very rapidly by non-radiative internal conversion towards the state S₁₀. The direct and very rapid decay of this state to one of the states S₀, so-called fluorescence, forms the main component of the emitted light, also so-called fast component.

Through non-radiative transitions, so-called inter-band crossing, singlet states can also be converted into spin triplet states S=1 (T₀, T₁, T₂, . . . ) and their vibrational states which will be de-exited by non-radiative conversion towards the state T₁₀. The decay from this state to one of the states S₀, after a much longer lifetime, is called phosphorescence. The states T₁ have a lower energy than the states S₁, the wavelengths of the phosphorescence spectrum will be longer than those of the fluorescence spectrum. The phosphorescence mechanism does not intervene in the detection of ionising radiations.

Some molecules in the triplet state T₁ may be re-excited to the state S₁ by triplet-triplet annihilation between two excited molecules of the triplet states: one of the molecules transfers its excitation energy to the other, resulting in a molecule returning to the fundamental state and the other is raised to a singlet state S₁ with a higher energy. Afterwards, this state S₁ decays towards the states S₀ by fluorescence. This is delayed fluorescence and forms the slow component of the emitted light which plays an essential role in the discrimination of the different radiations.

Hence, the mechanisms involved in the detection are the rapid fluorescence (a duration from a few ns to a few μs) and the delayed fluorescence (a duration from a few hundreds of ns to a few min), respectively forming the fast and slow components of the emitted light.

Transition into a triplet state is much less likely than into a singlet state and requires a high activation density of the ionising particle. Hence, the ratio between the fast and slow components is proportional to the energy loss dE/dx along the trajectory which depends on the nature of the ionising particle: very low for electrons, large for charged particles and increasing with their mass.

The discrimination between gammas, neutrons and light charged particles (alphas, etc.) is then obtained by analysis of the shape of the output signal.

The detection of neutral radiations such as gammas and neutrons poses an additional difficulty compared to that of charged particles because they do not interact directly with the material and have first to undergo one interaction producing detectable charged particles. The gamma rays interact with the medium by photoelectric effect, Compton effect or by creation of pairs by transferring energy to the electrons of the medium. As regards neutrons, different processes take place according to their energy. The fast neutrons (E≥100 keV) interact essentially with the protons (¹H) of the medium by elastic diffusion. The slow neutrons (E≤1 keV), for which the recoil nucleus resulting from the elastic diffusion is too slow to be detectable, have to undergo interactions leading to the production of charged particles.

The scintillators that are currently used for the detection of fast neutrons come essentially in three types:
liquid scintillators (NE213, BC501, BC501A): mixtures of xylene and naphthalene to which additives are added. Although very effective, these liquids have many drawbacks: they are toxic, corrosive, flammable, explosive, carcinogenic and hazardous for the environment and are therefore increasingly undesirable.
crystalline scintillators: organic and inorganic crystals (anthracene, stilbene, fluorophores doped with cerium). They are difficult to manufacture in large volumes, are very expensive and some are toxic. In addition, their response is a function of the angle of incidence of the incident particle, which might affect the detection response.
scintillating plastics: they are low-cost but do not generally allow neutron-gamma discrimination and can then be used only with a time reference for separating the two particles, this reference not being always available particularly in industrial applications. In addition, plastics cannot withstand temperatures beyond 70° C. A quiet recent development has led to the commercialisation by the company Eljen Technology of a plastic scintillator (EJ299) allowing for interesting neutron-gamma discrimination. A scintillator based on a similar technology, P77, had been developed by F. D. Brooks et al. in the 1960s. Yet, it has been recalled from the market because of the difficulty of synthesising it into a homogeneous and transparent material. In addition, as it ages, it becomes opaque and its geometry is deformed. The presence of PPO in a large amount in the initial matrix is done to the detriment of the mechanical and thermal resistance. Recent developments tend to demonstrate an improvement in these recurring problems in plastic matrices. However, for the moment, the scintillating liquid BC501 or the like remains the favourite material in fundamental research.

As regards slow neutrons, most commonly used detectors are based on ³He, ⁶Li and ¹⁰B. In these cases, the neutron undergoes a nuclear reaction and the products of the reaction are detected.

### OBJECTS OF THE INVENTION

To date, the detection of neutrons has been based on different interaction principles according to their energy and therefore allows implementing different detectors. The present invention aims to solve the technical problem consisting in providing a compound or a material capable of detecting both fast (E≥100 keV) and slow (E≤1 keV) neutrons by discriminating the gamma rays with the same scintillator. The present invention aims to solve the technical problem consisting in providing compounds allowing accessing the two interaction types in the same material while limiting and preferably while avoiding a trade-off on the efficiency of detection of neutrons of different energies.

The present invention aims to solve the technical problem consisting in providing a candidate for replacing ³He. The object of the present invention is to solve the technical problem consisting in providing an ionic compound or material having excellent thermal stability (up to 200° C.) and chemical stability. The present invention aims to solve the technical problem consisting in providing an ionic compound or material with no vapour pressure, and therefore non-flammable, unlike neutral organic compounds. Similarly, the present invention aims to associate a cation and an anion, both active in detection.

The present invention also aims to solve the technical problem consisting in providing a pure solid ionic compound or material (100% of the active molecule), which is not the case with existing scintillating materials. The present invention aims to solve the technical problem consisting in providing a compound or material allowing considering a miniaturisation of the detector and using it in severe environments, including use thereof under vacuum. The present invention aims to solve the technical problem consisting in providing a compound or material that is potentially usable in numerous applications relating to neutron detection.

Thus, the invention aims to solve the above-mentioned technical problems, while providing compounds that are simple to be synthesised.

The invention also aims to provide new molecules allowing improving the detection of the gamma, X, neutron, proton and alpha radiations, and possibly heavier particles.

The present invention also aims to provide new molecules allowing improving the discrimination of the gamma, X, neutron and lightly charged particles radiations as well as detecting neutrons regardless of their energy.

In particular, the present invention aims in particular to improve the properties of real-time discrimination of the neutron and gamma radiations of the compounds synthesised before according to the method described in the application WO 2014/147078.

### DESCRIPTION OF THE INVENTION

The Inventors have discovered a new family of discriminating ionic molecules (or compounds). The synthesis method allows obtaining imidazolium (salts consisting of a luminescent cation and an anion that is active in detection) in two or three steps from commercial products. This synthesis method is universal because a large number of aryls or aromatic groups can be coupled.

Moreover, the properties of these new molecules are even better with regards to the detection and discrimination of the aforementioned rays than those described in the patent WO 2010/004228.

The present invention relates to a compound or a material comprising it, said compound having one of the following ionic chemical structures:

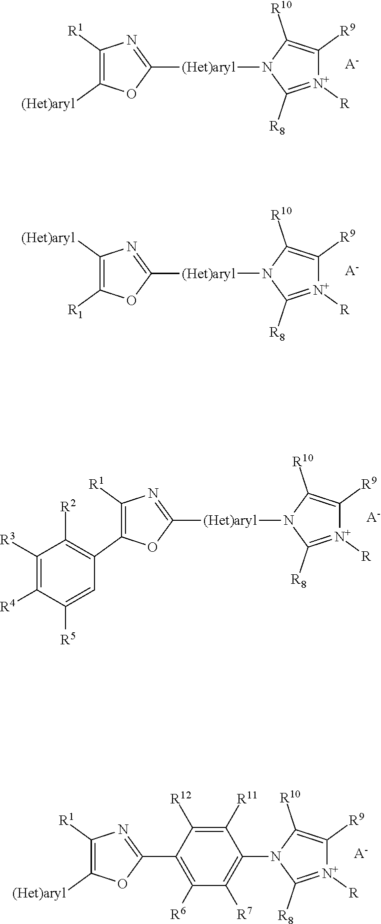

“R” represents an “alkyl” or O-“alkyl” group, “alkyl” being defined as a saturated hydrocarbon radical, possibly comprising one or more unsaturation(s), in a linear or branched chain, having 1 to 30 carbon atoms, preferably 3 to 18 carbon atoms, possibly substituted; R1, R2, R3, R4, R5, R6, R7, R8, R9, R10, R11, and R12 represent independently of one another an atom or group of atoms;
“(Het)Aryl” independently represents an aryl or heteroaryl group; and
A represents an anion.

In particular, the present invention relates to a compound or a material comprising it, said compound having one of the following ionic chemical structures:

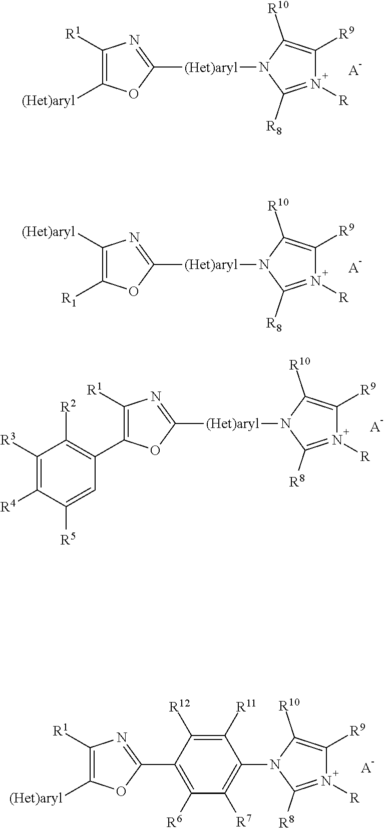

“R” represents an “alkyl” or O-“alkyl” group, “alkyl” being defined as a linear or branched chain saturated hydrocarbon radical having 1 to 30 carbon atoms, preferably 3 to 18 carbon atoms, possibly substituted;
R1, R2, R3, R4, R5, R6, R7, R8, R9, R10, R11, and R12 represent independently of one another an atom or group of atoms;
“(Het)Aryl” independently represents an aryl or heteroaryl group; and
A⁻ represents an anion.

According to one variant, a compound according to the invention has the following chemical structure:

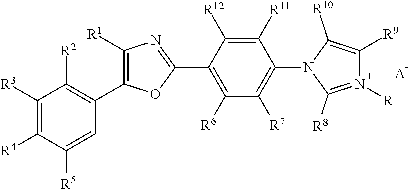

where R, R1, R2, R3, R4, R5, R6, R7, R8, R9, R10, R11, and R12 are as defined according to the invention.

According to one variant, R1, R2, R3, R4, R5, R6, R7, R8, R9, R10, R11, and R12 represent, independently of one another:
H,
F, Cl, Br, I,
a C₁-C₃₀ alkyl group, C₃-C₇ cycloalkyls, a C₆-C₁₀ aryl, a heteroaryl, an aralkyl, an heteroarylalkyl, wherein said alkyl or aryl groups are possibly substituted with 1 to 3 R²⁰ groups,
R²⁰ is selected from among OR²², NR²³R²⁴, NHOH, NO₂, CN, CF₃, a C₁-C₆ alkyl, a C₂-C₆ alkenyl, a C₂-C₆ alkynyl, a C₆-C₁₀ aryl, an aralkyl, ═O, C(═O)R²², CO₂R²², OC(═O)R²², C(═O)NR²³R²⁴, OC(═O)NR²³R²⁴, NR²¹C(═S)R²² or S(O)_{y}R²²;
R²² is, at each occurrence, independently selected from among H, a C₁-C₃₀-alkyl, preferably a C₅-C₂₀ alkyl, a C₆-C₁₀ aryl and an aralkyl;
R²³ and R²⁴ are, at each occurrence, independently selected from among H, a C₁-C₃₀, preferably C₅-C₂₀, alkyl and a C₆-C₁₀ aryl, or R²³ and R²⁴ form, with the hydrogen atom to which they are attached, a 3- to 7-membered heterocyclic group.

According to one variant, R1, R2, R3, R4, R5, R6, R7, R8, R9, R10, R11, and R12 represent, independently of one another, a hydrogen atom, a halogen atom, a C₁-C₆ alkyl group, a C₂-C₆ alkenyl, a C₂-C₆ alkynyl, a C₆-C₁₀ aryl, a C₆-C₂₀ aralkyl.

According to a variant of the present invention, the alkyl radicals represent saturated hydrocarbon radicals, in a linear or branched chain, having 1 to 30 carbon atoms, preferably 5 to 20 carbon atoms.

According to one variant, “R” represents an “alkyl” or an O-“alkyl” group, where “alkyl” is a saturated hydrocarbon radical, possibly comprising one or more unsaturation(s), in a linear or branched chain, having 5 to 12 carbon atoms.

According to a preferred variant, “R” represents an “alkyl” group or O-“alkyl” group, where “alkyl” is a saturated hydrocarbon radical, in a linear or branched chain, having 5 to 12 carbon atoms.

According to one variant, “R” represents an alkyl group CₙH₂ₙ₊₁ or O—CₙH₂ₙ₊₁, where n is a number ranging from 1 to 30, and preferably from 5 to 20.

Preferably, “R” is selected from among the group consisting of methyl, ethyl, propyl, butyl, pentyl, hexyl, octyl, nonyl, decyl, dodecyl, tetradecyl, pentadecyl, hexadecyl, heptadecyl, octadecyl, isopropyl, tert-butyl, 2-ethylhexyl, 2-methylbutyl, 2-methylpentyl, 1-methylpentyl and 3-methylheptyl radicals. Mention may in particular be made of the methyl, ethyl, propyl, butyl, pentyl, hexyl, octyl, nonyl, decyl, dodecyl, hexadecyl and octadecyl radicals when they are linear.

Mention may in particular be made of the isopropyl, tert-butyl, 2-ethylhexyl, 2-methylbutyl, 2-methylpentyl, 1-methylpentyl and 3-methylheptyl radicals, when they are branched or substituted with one or more alkyl radical(s).

Throughout the description of the invention, unless stated otherwise, the following terms should be understood as having the following meanings.

The term “haloalkyl” refers to an alkyl radical substituted with one or more halogen atom(s). Haloalkyl radicals include perhaloalkyl radicals and in particular perfluoroalkyl radicals of formula CₙF₂ₙ₊₁.

The term “halogen” refers to a chlorine, bromine, iodine or fluorine atom.

The term “cycloalkyl” means a mono- or multicyclic non-aromatic ring system having 3 to 10 carbon atoms, preferably 5 to 7 carbon atoms. As example of monocyclic cycloalkyl, mention may in particular be made of cyclopentyl, cyclohexyl, cycloheptyl and the like. As example of a multicyclic cycloalkyl group, mention may in particular be made of 1-decalin, norbornyl, or adamant-(1 or 2)-yl.

The term “alkenyl” refers to an aliphatic hydrocarbon group which contains a carbon-carbon double bond and which may be linear or branched having 2 to 6 carbon atoms in the chain. Branched means that one or more lower alkyl group(s), such as methyl, ethyl or propyl, are bonded to a linear alkenyl chain. As example of an alkenyl group, mention may in particular be made of ethenyl, propenyl, n-butenyl, i-butenyl, 3-methylbut-2-enyl or n-pentenyl.

The term “alkynyl” refers to an aliphatic hydrocarbon group which contains a carbon-carbon triple bond and which may be linear or branched having 2 to 6 carbon atoms in the chain, preferably 2 to 4 carbon atoms. Branched means that one or more lower alkyl group(s), such as methyl, ethyl or propyl, are bonded to a linear alkynyl chain. As example of alkynyl groups, mention may in particular be made of ethynyl, propynyl, n-butynyl, 2-butynyl, 3-methylbutynyl, and n-pentynyl.

The term “aryl” refers to an aromatic monocyclic or multicyclic ring system having 5 to 20 carbon atoms. As example of aryl groups, mention may in particular be made of phenyl, possibly substituted, naphthalene, possibly substituted, fluorene, possibly substituted, methylcarbazole, possibly substituted, and anthracene, possibly substituted, pyrene, possibly substituted, tetracene, possibly substituted, bodipy, thiophene and polythiophene.

The term “substituted” refers to a substitution of a hydrogen atom with a halogen atom or with an alkyl, haloalkyl, cycloalkyl, alkenyl, alkynyl, aryl group, a heterocyclic group, such as a heterocycloalkyl, a heteroaryl.

The term “aralkyl” refers to an aryl-alkyl-group, in which the aryl and the alkyl are as described in the present document. As example of aralkyl groups, mention may be in particular be made of benzyl, 2-phenethyl and naphthylmethyl.

The term “heterocyclic group” refers to a substituted or unsubstituted, mono- or multicyclic carbocyclic group in which the cyclic part comprises at least one heteroatom such as O, N, S or B. The nitrogen and the sulphur may possibly be oxidised, and the nitrogen may possibly be substituted in the aromatic rings. The heterocyclic groups comprise the heteroaryl groups and the heterocycloalkyl groups.

The term “heterocycloalkyl” refers to a cycloalkyl group in which one or more ring carbon atom(s) is/are substituted with at least one atom selected from among O, N, or S.

As example of a heterocycloalkyl group, mention may in particular be made of pyrrolidinyl, pyrrolinyl, imidazolidinyl imidazolinyl, pirazolidinyl, pirazolinyl, pyrazalkinyl, piperidyl, piperazinyl, morpholinyl, thiomorpholinyl, tetrahydrofuranyl, dithiolyl, oxathiolyl, oxadiazolyl, oxathiazolyl, pyranyl, oxazinyl, oxathiazinyl, and oxadiazinyl.

The term “heteroaryl” or “heteroaromatic” refers to a ring system containing from 5 to 10 carbon atoms in which at least one ring carbon is replaced by at least one atom selected from among —O—, —N—, —S—, or B. As example of a heteroaryl group, mention may in particular be made of pyrrolyl, furanyl, thienyl, pirazolyl, imidazolyl, thiazolyl, isothiazolyl, isoxazolyl, oxazolyl, oxathiolyl, oxadiazolyl, triazolyl, oxatriazolyl, furazanyl, tetrazolyl, pyridyl, pyrazinyl, pyrimidinyl, pyridazinyl, triazinyl, indolyl, isoindolyl, indazolyl, benzofuranyl, isobenzofuranyl, purinyl, quinazolinyl, quinolyl, isoquinolyl, benzoimidazolyl, benzothiazolyl, benzothiophenyl, thianaphthenyl, benzoxazolyl, benzisoxazolyl, cinnolinyl, phthalazinyl, naphthyridinyl, and quinoxalinyl. Also covered by the definition of “heteroaryl” groups, the fused ring systems including in particular the ring systems in which the aromatic ring is fused with a heterocycloalkyl ring. As examples of such fused ring systems, mention may in particular be made of phthalamide, phthalic anhydride, indoline, isoindoline, and tetrahydroisoquinoline.

The term “heteroarylalkyl” refers to an aryl-heteroaryl-group, in which the heteroaryl and the alkyl are as described in the present document.

The term “(Het)Ar” or “(Het)Aryl” or “(Het)Aromatic” refers to both an aryl and an heteroaryl group.

The terms “alkylene, cycloalkylene, heterocycloalkylene, arylene, heteroarylene” respectively refer to divalent alkyl, cycloalkyl, heterocycloalkyl aryl and heteroaryl groups, these groups being as defined hereinabove.

The term “metallate” refers to an anionic complex containing a metal, in particular a transition metal complexed by several ligands, for example a chalcogen such as oxygen or a cyanide group. Preferably, the metallate anion is a cyanometallate or oxometalate group.

The term “carborane” refers to an anionic molecule consisting of boron, carbon and hydrogen atoms and carrying a negative charge. As example, mention may be made of CB₁₁H₁₂.

The term “halide oxide” refers to oxides of formula HalOₓ⁻ where Hal represents Br, Cl or I and x is an integer from 1 to 4. As example, mention may be made of ClO₄⁻, IO₃⁻. The term “carbanion” refers to a compound comprising a carbon atom carrying a negative charge. As example, mention may be made of (CF₃SO₂)₃C⁻.

According to one variant, A⁻ is an anion selected from among a halide, P(R⁴)₆⁻, B(R⁴)₄⁻, SCN⁻, (R⁵SO₂)₂N⁻, R⁵OSO₃, R⁵SO₃⁻, carborane, carbonate, hydrogencarbonate, alcoholate, carboxylate, amidure, phosphate, SiF₆⁻, SbF₆⁻, I₃⁻, nitrate, halide oxide, silicate, sulphate, sulphonate, cyanide, carbanion, or metallate, wherein:
R⁴ is, at each occurrence, a group independently selected from among a halogen atom, a C₁-C₆ alkyl group, a C₆-C₁₀ aryl group, an aralkyl group;
R⁵ is, at each occurrence, a group independently selected from among a C₁-C₂₀ alkyl, a C₁-C₂₀ haloalkyl, a C₆-C₁₀ aryl, an aralkyl.

According to one variant, A⁻ (anion) is selected from among Cl⁻, Br⁻, I⁻, PF₆⁻, BF₄⁻, (CF₃SO₂)₂N⁻.

According to one variant, A⁻ (anion) represents PF₆⁻ or BF₄⁻.

According to a particular embodiment, the compound according to the present invention has the following ionic structure associating a luminescent cation with an anion that is active in the slow neutron detection:

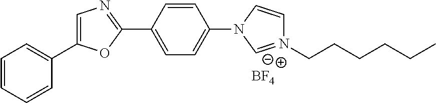

Preferably, the fluorophore compound is a compound as defined according to the invention, R comprising all of the variants and embodiments, and combinations thereof.

According to one embodiment, the compound according to the invention is solid under the conditions of use, and in particular at 20° C. and 101 325 Pa. Advantageously, the compound according to the invention is solid up to 200° C. at 101 325 Pa.

Advantageously, the invention relates to a luminescent ionic compound, preferably solid, allowing detecting both low and high energies neutrons by discriminating them from gammas.

According to a second aspect, the invention relates to a solid or liquid fluorophore material, comprising or consisting of a fluorophore compound, said fluorophore compound being defined according to the invention.

Advantageously, the material according to the invention is a transparent plastic material.

According to another aspect, the invention relates to the use of these molecules for their fluorophore properties.

Advantageously, the invention relates to the use of a compound or of a material as defined according to the invention for the detection of gamma, X, neutron, proton radiations.

Advantageously, the invention relates to the use of a compound or of a material as defined according to the invention for the discrimination of proton/gamma, proton/X, neutron/gamma, neutron/X, alpha/gamma, alpha/X radiations.

Advantageously, the invention relates to the use of a compound or of a material as defined according to the invention for the manufacture of a radio-detection, industrial or medical dosimetry instrument.

Advantageously, the invention relates to a compound and a material with a high detection efficiency and neutron detection regardless of their energy and a very good discrimination between neutrons and gammas.

Advantageously, the invention relates to a new generation compound and material having a better neutron/gamma discrimination merit factor, a high intrinsic detection efficiency, a strong luminescence in the solid state and a better thermal stability (advantageously up to 200° C.) compared to the fluorene-type material (100° C.) described by the application WO 2014/147078.

As far as the Inventors know, none of the existing competing products allows detecting both fast and slow neutrons in a satisfactory manner. Their intrinsic detection efficiency is also lower than that of the compounds according to the present invention, as well as the emitted light.

Advantageously, the invention relates to a compound and a material having a high chemical, thermal stability, without vapour pressure and allowing using it under vacuum.

Advantageously, the invention relates to a solid compound and material allowing using it in sensitive locations.

Advantageously, the invention relates to a compound the synthesis of which can be industrialised at least on the kilogram scale.

Advantageously, the invention relates to a compound in the form of crystals, and in particular single crystals, for example having a centimetric size or more.

The present invention may be implemented for the detection of neutrons both in the military and civil fields, some applications being common to both. A list of possible applications is given hereafter:
1) radiation monitoring on workplaces and in the environment
2) fissile material monitoring in nuclear wastes
3) detection of fissile materials in suspicious packages
4) nuclear diagraphy
5) R&D for nuclear energy
6) cosmic radiation study, cosmic ray monitoring in aircrafts
7) neutron radiography
8) physics fundamental research

Advantageously, the invention relates to a compound and a material for the manufacture of a scintillator containing only organic molecules synthesised from easily accessible raw materials.

Advantageously, the invention relates to a compound and a material offering the possibility of miniaturising the devices and making them portable.

Furthermore, advantageously, the invention relates to a compound and a material allowing for a reduction in the costs to a few tens of euros per gram compared to about 15 k€ the gram for ³He.

The needs for low-energy neutrons (slow neutrons) are covered essentially by ³He gaseous detectors, the marketing of which will be no longer possible in few years given the rarefaction of ³He on Earth. These devices detect only low-energy neutrons.

According to another aspect, the present invention also relates to a synthesis method allowing preparing compounds according to the invention.

In particular, the invention relates to a method for preparing a compound comprising an imidazolium group bonded directly to an aromatic group by a covalent bond between a nitrogen atom of the imidazolium group and a carbon atom of said aromatic group, said method comprising a reaction of coupling an aromatic group to an imidazole group by creating a covalent bond between an sp² carbon atom of the aromatic group and a nitrogen atom of the imidazole group, in the presence of a NaY zeolite including copper (II) and a base, preferably by Ullmann-type coupling.

According to a particular embodiment, the invention relates to the preparation of a compound of formula (I) according to the invention, said method comprising:
(i) a reaction of coupling a “(Het)Ar” aromatic group to an imidazole group by creating a covalent bond between an sp² carbon atom of the aromatic group and a nitrogen atom of the imidazole group, in the presence of a NaY zeolite including copper (II) and a base, preferably by Ullmann-type coupling; and
(ii) a reaction of coupling an “R” group comprising at least one sp³ carbon atom with an imidazole group by creating a covalent bond between the carbon atom sp³ of the “R” group and a nitrogen atom of the imidazole group, preferably by nucleophilic substitution, preferably using a halogenated reactive “R” group.

According to one variant, step (i) corresponds to the following reaction:

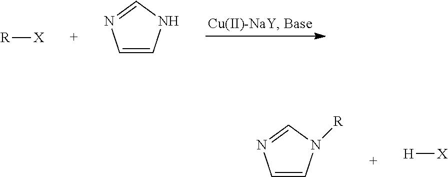

where X is a halogen atom, preferably the reaction being carried out in the presence of microwave radiations.

Typically, according to the invention, the “R—X” group represents the structure:

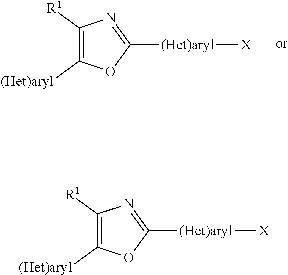

The catalyst is simple to prepare: the NaY zeolite is placed in the presence of copper sulphate dissolved in water and stirred for 24 hours. The zeolite impregnated with copper (II) is recovered by filtration, dried in the oven (100° C.) and then calcined at 550° C. for 4 hours. Reference may in particular be made to the publication by M. L. Kantam, B. P. C. Rao, B. M. Choudrary, R. S. Reddy, Synlett, 2006, 14, 2195-2198. doi: 10.1055/s-2006-949615.

During the Ullmann coupling (J. Hassan, M. Sevignon, C. Gozzi, E. Schulz, M. Lemaire, “Aryl-Aryl Bond Formation One Century after the Discovery of the Ullmann Reaction”, Chemical Reviews, vol. 102, 2002, p. 1359-1470), the reagents are heated, for example, in a tube sealed for 72 hours at 180° C. using a sand bath. To recover the reaction crude, all it needs is to carry it on in an organic solvent such as dichloromethane and to filter in order to remove the zeolite and the base (potassium carbonate). The imidazole derivative thus obtained is purified, for example, by column chromatography.

Although it does not use a solvent or an inert atmosphere, it is necessary to heat the reaction mixture necessary for coupling of the imidazole and the aryl, for example by heating to a temperature of 180° C. for 72 hours.

Advantageously, the reaction is carried out in the presence of microwave radiations. By this method, the Inventors have been able to reduce the reaction time quite substantially. It is possible to reduce a reaction time from 72 hours (in a sealed tube heated with a sand bath) to less than 3 hours and that being so for identical yields.

Microwaves are electromagnetic waves that have a wavelength comprised between 30 cm (1 GHz) and 1 mm (300 GHz).

Preferably, the base is selected from among a potassium or cesium carbonate, a potassium phosphate, a cesium phosphate, LiOH, NaOH, or KOH.

Advantageously, the method according to the present invention does not require any solvent. According to the prior art, the synthesis methods require the use of a solvent (dimethylformamide, dimethyl sulfoxide, toluene, etc.), which is avoided in the present invention.

The present invention allows avoiding the use of amine ligands (carbene, phenanthroline, I-proline, etc.).

This method allows avoiding the use of solvents, of an inert atmosphere and of expensive ligands, and allows preparing the compounds of the invention in only two or three steps. Moreover, the method according to the invention allows using commercially-available molecules.

Quite advantageously, the use of microwave radiations during the Ullmann-type coupling allows significantly reducing the reaction time. Thus, the reaction time can switch from 72 to less than 3 hours.

The first synthesis step consists in carrying out the Ullmann-type coupling, between an imidazole, i.e. a molecule comprising an imidazole group, and a molecule bearing a halogenated aromatic group, i.e. bearing a halogen atom on a carbon atom, a halogen atom which will be replaced by imidazole.

The second step consists in making the imidazole thus obtained react with an “R—X” molecule to create a covalent bond between the imidazole and an sp³ carbon atom of this molecule.

According to one variant, this second step consists in carrying out an N-alkylation, i.e. making the imidazole react with an alkyl radical to create an N-alkyl bond between the alkyl radical and the nitrogen atom of the imidazole (nitrogen atom bearing no aromatic group grafted by Ullmann coupling).

A possible third step consists in exchanging the halide anion in order to obtain a fluorophore compound. This third step may be carried out in a biphasic medium.

It is possible to use the potassium, lithium, or sodium salt of the desired anion.

Possibly, said method may also comprise the step consisting in isolating the obtained product.

The method of the invention is particularly interesting as it can be extrapolated on an industrial scale because of the small number of synthesis steps, of the used reagents, which are commercial, as well as the equipment for the reactions, which can be used on a larger scale for industrial reactions.

In particular, the invention covers devices comprising the compounds of the invention, suitable for use in nuclear power plants, hospitals, airports, or similar locations.

The compounds of the invention having no measurable vapour tension can be used under vacuum.

The specific applications of the compounds of the invention depend on the considered molecule and vary according to the nature of the anion, of the cation (initial imidazole) and of the aromatic group, which must in particular serve as a fluorophore.

One could consider that the use of compounds of the invention allows lowering the detection threshold and the sensitivity of existing installations in the fields such as nuclear safety (radioprotection, proliferation of nuclear wastes or weapons, nuclear reactor) and fundamental research.

To characterise this product and verify its purity, conventional analyses methods such as nuclear magnetic resonance (NMR), infrared (IR), ultraviolet/visible (UV/visible) spectroscopy and analyses have been carried out.

These techniques have been used to characterise the synthesised compounds.

Advantageously, the compounds of the invention may be used pure, i.e. in the form of a material consisting essentially of at least one compound of the invention. Advantageously, a high concentration allows increasing as much the radiation detection efficiency. The previously-described compounds (NE213 or BC501) do not enable the use of high concentrations because their radiation detection power decreases starting from a given concentration. Thus, in this respect, the invention is a major improvement. The invention also covers the use of the compounds of the invention in a diluted form, as a filler in polymer matrices, as components of composite materials. The concentration of the compounds of the invention may be from 1 to 80% by weight in a polymer or in a solvent.

### Example

### Example 1—Procedure for the Synthesis of N—POP-imidazole (2-(4-(1H-imidazol-1-yl)phenyl)-5-phenyloxazole) by Ullmann Type Coupling

2-(4-bromophenyl)-5-phenyloxazole (POPBr) (71.5 g, 238 mmol), imidazole (24.32 g, 357.3 mmol), potassium carbonate (65.84 g, 476.4 mmol) and NaY zeolite doped with copper at 8% are ground together using a mortar. The obtained powder is introduced into a 800 ml Teflon microwave reactor equipped with a magnetic stirring blade. The medium is heated up to 210° C. for 3 h. After cooling, the obtained solid is ground into fine powder and then extracted with dichloromethane. Filtration on Celite allows obtaining the product, which is then purified by conventional laboratory techniques. The N—POP-imidazole is obtained in the form of a white powder (64.1 g, 223.1 mmol, 93%).

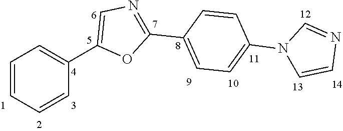 [SMILES: c1ccc(-c2cnc(-c3ccc(-n4ccnc4)cc3)o2)cc1]

¹H NMR (500 MHz, Chloroform-d): δ=8.21 (AA′, ³J_{H9-H10}=8.7 Hz, 2H, H₉), 7.94 (s, 1H, H₁₂), 7.72 (d, ³J_{H3-H2}=8.0 Hz, 2H, H₃), 7.50 (BB′, ³J_{H10-H9}=8.7 Hz, 2H, H₁₀), 7.46 (s, 1H, H6), 7.45 (t, ³J_{H2-(H3+1)}=7.8 Hz, 2H, H₂), 7.34 (tt, ³J_{H1-H2}=7.4 Hz, ⁴J_{H1-H2}=1.3 Hz, 1H, H₁), 7.35 (s large, 1H, H₁₃), 7.24 (s large, 1H, H₁₄).

¹³C NMR (125 MHz, Chloroform-d): δ=160.12 (C₇), 151.91 (C₅), 138.72 (C₈), 135.60 (large, Cₗₘ), 131.09 (Cₗₘ), 129.19 (C₁₀), 128.92 (C₁), 128.06 (C₉), 127.92 (C₄), 126.69 (C₁₁), 124.45 (C₃), 123.82 (C₆), 121.57 (C₂), 118.07 (br, Cₗₘ).

IR: νₘₐₓ/cm⁻¹=3107 (wide, ν_{C—H aromatic}), 1614, 1504, 1303, 1054, 951, 833, 767, 725, 691, 651, 492.

UV-Vis (MeCN): λₘₐₓ=316 nm, εₘₐₓ=38.000 L·mol⁻¹·cm⁻¹.

Element analysis for (C₁₈H₁₃N₃O₁). Calculated: C, 75.25; H, 4.56; N, 14.63. Found: C, 75.30; H, 4.58; N, 14.82.

### Example 2—Procedure for the Synthesis of N—POP—N′-hexylimidazolium bromide (3-hexyl-1-(4-(5-phenyloxazol-2-yl)phenyl)-1H-imidazol-3-ium bromide

N—POP-imidazole (40 g, 139 mmol) is suspended in acetonitrile (40 ml) and n-bromohexane (60 ml, 418 mmol) in an autoclave. The medium is stirred at 120° C. for 48 h; then, after cooling, the reaction medium is poured onto diethyl ether (500 ml). The product is recovered by filtration, and is washed again with 3 portions of 100 ml of diethyl ether. To achieve an optimum purity, the product is purified according to conventional laboratory techniques. The product is obtained in the form of a white powder (60.152 g, 133 mmol, 95%).

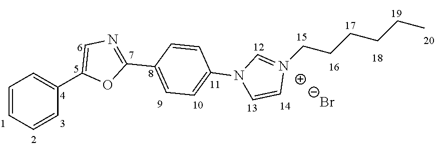 [SMILES: CCCCCC[n+]1ccn(-c2ccc(-c3ncc(-c4ccccc4)o3)cc2)c1 ; [Br-]]

¹H NMR (500 MHz, Chloroform-d): δ=11.04 (t, ³J_{H12-(H13+14)}=1.8 Hz, 1H, H₁₂), 8.16 (AA′, ³J_{H9-H10}=8.8 Hz, 2H, H₉), 8.07 (t, ³J_{H13-(H12+14)}=1.8 Hz, 1H, H₁₃), 8.00 (BB′, ³J_{H10-H9}=8.8 Hz, 2H, H₁₀), 7.73 (t, ³J_{H14-(H12+13)}=1.8 Hz, 1H, H₁₄), 7.60 (d, ³J_{H3-H2}=8.3 Hz, 2H, H₃), 7.37 (t, ³J_{H2-(H3+1)}=7.8 Hz, 2H, H₂), 7.36 (s, 1H, H₆), 7.29 (tt, ³J_{H1-H2}=7.4 Hz, ⁴J_{H1-H2}=1.2 Hz, 1H, H₁), 4.53 (t, ³J_{H15-H16}=7.3 Hz, 2H, H₁₅), 1.97 (m, 2H, H₁₆), 1.37 (m, 2H, H₁₇), 1.37 (m, 4H, H₁₈₊₁₉), 0.81 (t, 3H, ³J_{H20-H19}=7.1 Hz H₂₀).

¹³C NMR (125 MHz, Chloroform-d): δ=159.11 (C₇), 152.22 (C₅), 135.95 (C₁₂), 135.38 (C₈), 129.12 (C₂), 128.98 (C₁), 128.94 (C₄), 128.20 (C₉), 127.47 (C₁₁), 124.39 (C₃), 123.86 (C₆), 123.43 (C₁₄), 122.26 (C₁₀), 120.90 (C₁₃).

IR: νₘₐₓ/cm⁻¹=3523 (wide, ν_{O—H}); 3079 (ν_{C—H aromatic}); 2960, 2929, 2856 (ν_{C—H aliphatic}); 1551, 1498, 1206, 1135, 1059, 952, 842, 769, 737, 694, 623, 532, 493.

UV-Vis (MeCN): λₘₐₓ=321 nm, εₘₐₓ=33.200 L·mol⁻¹·cm⁻¹.

Element analysis (C₂₄H₂₆BrN₃O₁). Calculated: C, 63.72; H, 5.79; N, 9.29. Found: C, 62.76; H, 5.71; N, 9.29. The element analysis corresponds to the product containing 0.5 H₂O molecule per molecule, which is consistent with the presence of ν_{O—H} vibrations in the infrared spectrum (Calculated: C, 62.48; H, 5.90; N, 9.11).

### Example 3—Procedure for the Synthesis of N—POP—N′-hexylimidazolium tetrafluoroborate (3-hexyl-1-(4-(5-phenyloxazol-2-yl)phenyl)-1H-imidazol-3-ium tetrafluoro borate

N—POP—N′-hexylimidazolium bromide (23.7 g, 52.4 mmol) is dissolved at 50° C. in 350 ml of ethanol. Sodium tetrafluoroborate (8.63 g, 78.6 mmol) is dissolved in 50 ml of water and this solution is added to the imidazolium salt solution. The medium becomes slightly turbid and is stirred at room temperature for 1 h. Afterwards, water (1.5 L) is added in order to make the product precipitate, which is then recovered by filtration. The obtained white powder is dissolved in 500 ml of a 10% dichloromethane/methanol solution, then the obtained solution is dried over magnesium sulphate. The liquid is filtered, evaporated and then the obtained solid is purified according to conventional laboratory techniques in order to remove the remaining imidazolium bromide. N—POP—N′-hexylimidazolium tetrafluoroborate is obtained in the form of a microcrystalline white powder (22.737 g, 49.5 mmol, 94%).

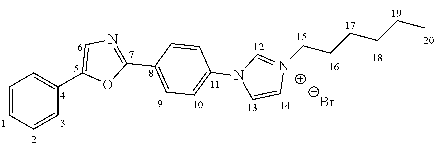 [SMILES: CCCCCC[n+]1ccn(-c2ccc(-c3ncc(-c4ccccc4)o3)cc2)c1 ; [Br-]]

¹H NMR (500 MHz, DMSO-d₆): δ=9.91 (t, ³J_{H12-(H13+14)}=1.7 Hz, 1H, H₁₂), 8.42 (t, ³J_{H13-(H12+14)}=1.8 Hz, 1H, H₁₃), 8.37 (AA′, ³J_{H9-H10}=8.8 Hz, 2H, H₉), 8.07 (t, ³J_{H14-(H12+13)}=1.7 Hz, 1H, H₁₄), 7.98 (BB′, ³J_{H10-H9}=8.8 Hz, 2H, H₁₀), 7.92 (s, 1H, H₆), 7.90 (d, ³J_{H3-H2}=8.0 Hz, 2H, H₃), 7.52 (t, ³J_{H2-(H3+1)}=7.7 Hz, 2H, H₂), 7.42 (tt, ³J_{H1-H2}=7.3 Hz, ⁴J_{H1-H2}=1.3 Hz, 1H, H₁), 4.46 (t, ³J_{H15-H16}=7.3 Hz, 2H, H₁₅), 1.91 (m, 2H, H₁₆), 1.32 (m, 6H, H₁₇₋₁₉), 0.88 (t, 3H, ³J_{H20-H19}=7.1 Hz H₂₀).

¹³C NMR (125 MHz, DMSO-d₆): δ=158.91 (C₇), 151.4 (C₅), 135.96 (C₈), 135.47 (C₁₂), 129.10 (C₂), 128.87 (C₁), 127.60 (C₄), 127.55 (C₉), 127.14 (C₁₁), 124.51 (C₆), 124.21 (C₃), 123.44 (C₁₄), 122.39 (C₁₀), 129.92 (C₁₃).

IR: νₘₐₓ/cm⁻¹=3148, 3101 (ν_{C—H aromatic}); 2957, 2928, 2859 (ν_{C—H aliphatic}); 1556, 1499, 1212, 1135; 1068 (wide, very intense, ν_{B—F}); 951, 840, 773, 737, 695, 625, 521, 494.

UV-Vis (MeCN): λₘₐₓ=321 nm, εₘₐₓ=33.100 L·mol⁻¹ cm⁻¹.

Element analysis (C₂₄H₂₆BF₄N₃O₁). Calculated: C, 63.76; H, 5.71; N, 9.15. Found: C, 62.78; H, 5.76; N, 9.21.

This compound is stable at 200° C.

This compound is highly luminescent (70%)

Measurements have been performed by irradiating the new materials with a fast neutron and gamma emitter AmBe source. The sample is placed on a photomultiplier PHOTONIS XP4512B which produces an electrical signal processed by digital electronics (FASTER developed in the Caen Corpuscular Laboratory (LPC)). Two integration gates allow matching the total and slow charges of the signal: a 300 ns gate covers the entire signal and a gate delayed by 50 ns and stopping at the same time as the total gate covers the slow component of the signal.

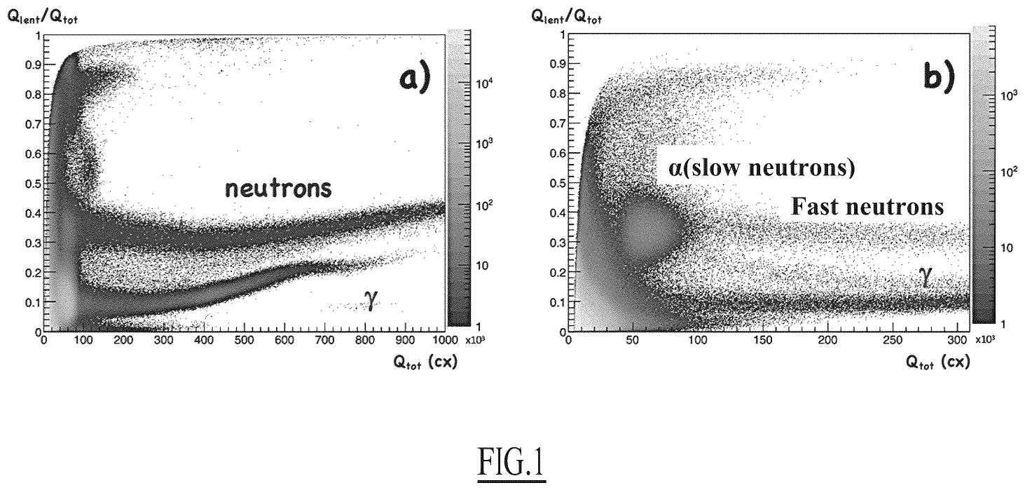

FIG. 1 shows, for the compound of Example 3, the correlations between the total charge Qₜₒₜ and the ratio Qₗₑₙₜ/Qₜₒₜ obtained by irradiation of the AmBe source:

FIG. 1a): an excellent discrimination between neutrons and gammas is obtained.

FIG. 1b): a polyethylene layer with a thickness of 10 cm placed in front of the detector allows slowing down the neutrons coming from the source. These, thermalised, will interact with the ¹⁰B and produce alphas. Not all neutrons are thermalised and a small fast neutron component subsists.

The peak observed in the gamma component (Qₜₒₜ≈60000 cx) of FIG. 1a) corresponds to the photopic resulting from the alpha disintegration of the ²⁴¹Am to an excited state of the ²³⁷Np, the return of which to the fundamental releases a gamma ray of 59.54 keV. This shows the very low energy threshold of this compound which allows detecting fast neutrons in an energy area (E<1 MeV) generally difficult to access by existing detectors.

A major feature of the neutron detectors is their discrimination between neutrons and gammas, determined by the figure of merit (FOM, Figure of merit). The compound shown in FIG. 1 has a FOM≥2, a very satisfactory value for separating the neutrons from the gammas.

### Example 4—Comparison of the Performances of a Material According to the Invention with a Fluorene-Based Material

The performances of the material obtained in Example 3 hereinabove have been compared with those of a 1(9H-fluoren-2-yl)-3-hexyl-1H-imidazol-3-ium tetrafluoro borate material, so-called “fluorene”-type compound of formula:

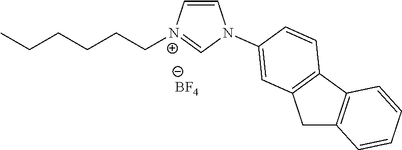

The compound of Example 3 differs from the “fluorene”-type compound by the substitution of the fluorene group with a 4-(5-phenyloxazol-2-yl)phenyl group, in particular by the substitution of the condensed cyclopentane of the “fluorene”-type compound with an oxazole group.

a) Irradiation with a UV (Ultraviolet) Radiation

The two samples are irradiated simultaneously by a UV light. It is observed that the material according to the invention is much more luminescent than the “fluorene”-type compound.

b) Irradiation with an AmBe Radiation

Measurements have been carried out by irradiating the two materials with a fast neutron and gamma emitter AmBe source. The sample is placed on a photomultiplier PHOTONIS XP4512B which produces an electrical signal processed by digital electronics (FASTER developed in the Caen Corpuscular Laboratory (LPC)). Two integration gates allow matching the total and slow charges of the signal: a 300 ns gate covers the entire signal and a gate delayed by 50 ns and stopping at the same time as the total gate covers the slow component of the signal.

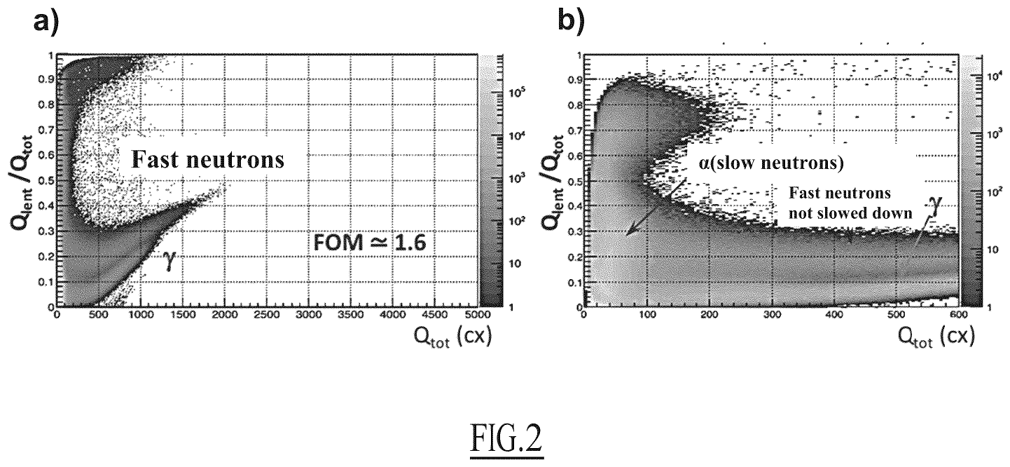

FIG. 2 shows, for the “fluorene”-type compound, the correlations between the total charge Qtot and the ratio Qlent/Qtot obtained by irradiation of the AmBe source.

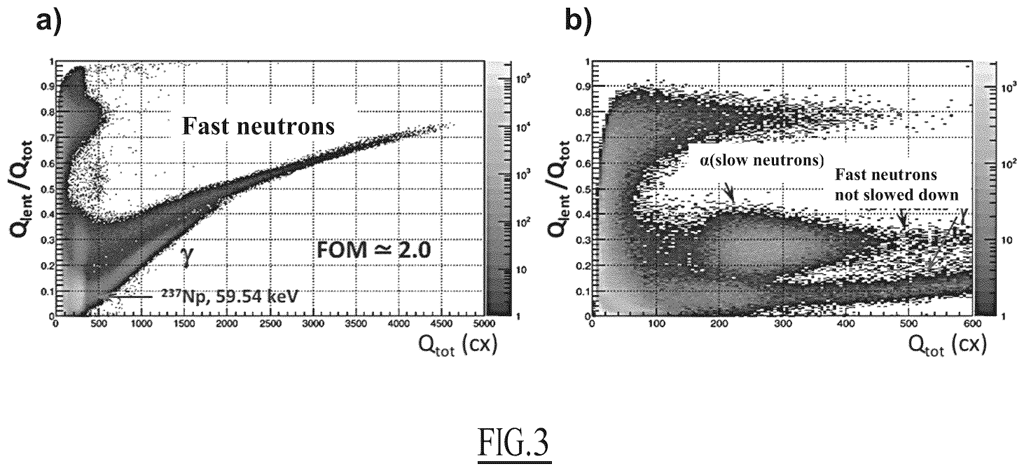

FIG. 3 shows for the compound of Example 3 the correlations between the total charge Qtot and the ratio Qlent/Qtot obtained by irradiation of the AmBe source.

FIGS. 2b) and 3b) have been obtained by placing a polyethylene layer having a thickness of 10 cm in front of the detector in order to slow down the neutrons coming from the source. These, thermalised, will interact with the 10B and produce alpha. Not all neutrons are thermalised and a small fast neutron component subsists.

By comparing FIGS. 2a) and 3a), it is observed that the discriminating power of the compound of Example 3 is substantially improved, compared to the “fluorene”-type material. In addition, the gain in luminescence yield associated with the compound of the invention is multiplied by 2, compared to the “fluorene” compound.

In addition, in the context of the compound of the invention, the ²³⁷Np photopic resulting from the α decrease of ²⁴¹Am is clearly visible, which demonstrates the possibility of obtaining lower detection thresholds and therefore also addressing the fast neutrons of lower energy.

By comparing FIGS. 2b) and 3b), it is observed in the case of the material of the invention that the α component produced by the ¹⁰B(n, α)⁷Li* reaction is substantially better separated from the background noise and from the γ component, with respect to the “fluorene” compound.

### Example 5—Luminescence Quantum Yield of a Material According to the Invention

The quantum yield of the material obtained in Example 1 hereinabove has been measured according to the following protocol:

The luminescence quantum yield measurements have been carried out from the material in the solid state, in the form of a polycrystalline powder. The measurements have been carried out using an integrating sphere system coupled to a multichannel photonic analyser (Hamamatsu Quantaurus).

The time-resolved measurements have been performed using the time-correlated single photon counting (TCSPC) electronics PicoHarp300 or multi-channel scaling (MCS) electronics NanoHarp 250 of the PicoQuant FluoTime 300 (PicoQuant GmbH, Germany), equipped with a laser pulse driver PDL 820. A pulsed laser diode LDH-P-C-375 (λ=375 nm, FWHM pulse <70 ps, repetition rate 200 kHz-40 MHz) has been used to excite the sample and mounted directly on the sample chamber at 90°. The photons have been collected by a photomultiplier detector (PMT) with single photon counting PMA-C-192.

The data have been acquired using the commercial software EasyTau (PicoQuant GmbH, Germany), whereas the analysis of the data has been performed using the commercial software FluoFit (PicoQuant GmbH, Germany).

The luminescence quantum yield measured from the compound obtained in Example 1 is equal to 67±2.5%, with a lifetime of 2 ns (excitation=290 nm, emission=407, 10 MHz).

The luminescence quantum yield of the compound of Example 3 according to the invention is about two times higher than the luminescence quantum yield associated with the “fluorene”-type compound. Thus, the fast neutron detection thresholds are significantly lowered in the context of the compound of Example 3, compared to the “fluorene”-type compound. Similarly, the alpha component resulting from the detection of slow neutrons can be distinguished more clearly from the background noise in the context of the compound of Example 3, compared to the “fluorene”-type compound.

1. A compound having one of the following ionic chemical structures: 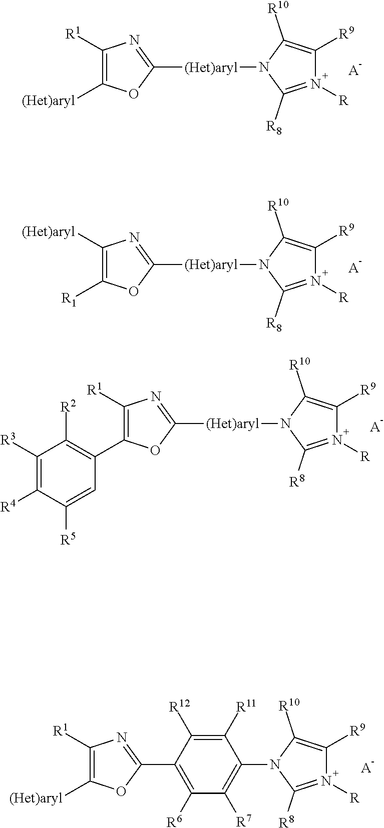
“R” represents an “alkyl” group or O-“alkyl” group, “alkyl” being defined as a saturated hydrocarbon radical, in a linear or branched chain, having 1 to 30 carbon atoms, possibly substituted;
R1, R2, R3, R4, R5, R6, R7, R8, R9, R10, R11, and R12 representing independently from each other an atom or group of atoms;
“(Het)aryl” independently represents an aryl or heteroaryl group; and
A⁻ represents an anion.

2. The compound according to claim 1, characterised in that it has the following chemical structure: 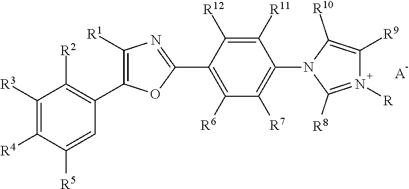
where R, R1, R2, R3, R4, R5, R6, R7, R8, R9, R10, R11, and R12 are as defined in claim 1.

3. The compound according to claim 1, characterised in that “R” represents an “alkyl” group or O-“alkyl” group, where “alkyl” is a saturated hydrocarbon radical, in a linear or branched chain, having 5 to 12 carbon atoms.

4. The compound according to claim 1, characterised in that “R” represents an alkyl group CₙH₂ₙ₊₁ or O—CₙH₂ₙ₊₁, where n is a number ranging from 1 to 30.

5. The compound according to claim 1, characterised in that “R” is selected from among the group consisting of methyl, ethyl, propyl, butyl, pentyl, hexyl, octyl, nonyl, decyl, dodecyl, tetradecyl, pentadecyl, hexadecyl, heptadecyl, octadecyl, isopropyl, tert-butyl, 2-ethylhexyl, 2-methylbutyl, 2-methylpentyl, 1-methylpentyl and 3-methylheptyl radicals.

6. The compound according to claim 1, characterised in that R1, R2, R3, R4, R5, R6, R7, R8, R9, R10, R11, and R12 represent independently of one another:
H,
F, Cl, Br, I,
a C₁-C₃₀ alkyl, a C₃-C₇ cycloalkyl, a C₆-C₁₀ aryl, heteroaryl, aralkyl, heteroarylalkyl, wherein said alkyl or aryl groups are possibly substituted with 1 to 3 R²⁰ groups,
R²⁰ being selected from among OR²², NR²³R²⁴, NHOH, NO₂, CN, CF₃, a C₁-C₆ alkyl, a C₂-C₆ alkenyl, a C₂-C₆ alkynyl, a C₆-C₁₀ aryl, an aralkyl, ═O, C(═O)R²², CO₂R²², OC(═O)R²², C(═O)NR²³R²⁴, OC(═O)NR²³R²⁴, NR²¹C(═S)R²² or S(O)_{y}R²²;
R²² is, at each occurrence, independently selected from among H, a C₁-C₃₀ alkyl, a C₆-C₁₀ aryl and an aralkyl;
R²³ and R²⁴ are, at each occurrence, independently selected from among H, a C₁-C₃₀, preferably C₅-C₂₀, alkyl, and a C₆-C₁₀ aryl, or R²³ and R²⁴ form with the hydrogen atom to which they are attached a 3- to 7-membered heterocyclic group.

7. The compound according to claim 1, characterised in that R1, R2, R3, R4, R5, R6, R7, R8, R9, R10, R11, and R12 represent independently of one another a hydrogen atom, a halogen atom, a C₁-C₆ alkyl, a C₂-C₆ alkenyl, a C₂-C₆ alkynyl, a C₆-C₁₀ aryl, a C₆-C₂₀ aralkyl.

8. The compound according to claim 7, wherein A⁻ is an anion selected from among halide, P(R⁴)₆⁻, B(R⁴)₄⁻, SCN⁻, (R⁵SO₂)₂N⁻, R⁵OSO₃, R₅SO₃⁻, carborane, carbonate, hydrogencarbonate, alcoholate, carboxylate, amidure, phosphate, SiF₆⁻, SbF₆⁻, I₃⁻, nitrate, halide oxide, silicate, sulphate, sulphonate, cyanide, carbanion, or metallate, wherein:
R⁴ is, at each occurrence, a group independently selected from among a halogen atom, a C₁-C₆ alkyl group, a C₆-C₁₀ aryl group, an aralkyl group;
R⁵ is, at each occurrence, a group independently selected from among C₁-C₂₀ alkyl, C₁-C₂₀ haloalkyl, C₆-C₁₀ aryl, aralkyl.

9. The compound according to claim 1, characterised in that A⁻ (anion) is selected from among Cl⁻, Br⁻, I⁻, PF₆⁻, BF₄⁻, (CF₃SO₂)₂N⁻.

10. The compound according to claim 1, characterised in that A⁻ (anion) represents PF₆⁻ or BF₄⁻.

11. A fluorophore solid or liquid material, comprising or consisting of a fluorophore compound, said fluorophore compound being defined according to claim 1.

12. The material according to claim 11, characterised in that the material is a transparent plastic material.

13. A use of a compound as defined according to claim 1, for the detection of the gamma, X, neutron, proton radiations.

14. The use according to claim 13, for the discrimination of the proton/gamma, proton/X, neutron/gamma, neutron/X, alpha/gamma, alpha/X radiations.

15. A use of a compound as defined according to claim 1, for the manufacture of a radio-detection, industrial or medical dosimetry instrument.

16. A method for preparing a compound of formula (I) as defined according to claim 1, said method comprising:
(i) a coupling reaction of an aromatic “(Het)Ar” group to an imidazole group by creating a covalent bond between an sp² carbon atom of the aromatic group and a nitrogen atom of the imidazole group, in the presence of a NaY zeolite including copper (II) and a base; and
(ii) a coupling reaction of an “R” group comprising at least one sp³ carbon atom to an imidazole group by creating a covalent bond between the sp³ carbon atom of the “R” group and a nitrogen atom of the imidazole group.
